(19) United States
(12) Patent Application Publication
(10) Pub. No.: US 2025/0084381 A1
(43) Pub. Date: Mar. 13, 2025

(54) MANUFACTURING DEVICE OF ARTIFICIAL SKIN USING CELLS DIFFERENTIATED FROM INDUCED PLURIPOTENT STEM CELLS

(71) Applicant: CLECELL, CO. LTD., Gyeonggi-do (KR)

(72) Inventors: Hyung Min Chung, Seoul (KR); C-Yoon Kim, Seoul (KR); Seul-Gi Lee, Seoul (KR); Gujin Chung, Seoul (KR)

(73) Assignee: CLECELL, CO. LTD., Gyeonggi-do (KR)

(21) Appl. No.: 18/280,218

(22) PCT Filed: Jun. 29, 2023
(86) PCT No.: PCT/KR2023/009097

§ 371 Date: Sep. 2, 2023

(30) Foreign Application Priority Data
Jul. 6, 2022 (KR) 10-2022-0083336

(51) Int. Cl. C12N 5/071; B33Y 30/00; B33Y 80/00; C12M 1/12; C12M 1/26; C12M 3/00

(52) U.S. Cl. CPC C12N 5/0698; B33Y 30/00; B33Y 80/00; C12M 21/08; C12M 25/06; C12M 33/04; C12N 2500/32; C12N 2501/11; C12N 2501/155; C12N 2506/45; C12N 2513/00

(57) ABSTRACT

The present invention relates to artificial skin using fibroblasts and keratinocytes, and more particularly, to a manufacturing device of artificial skin using cells differentiated from induced pluripotent stem cells (iPSCs). To this end, there is provided a manufacturing device of artificial skin including: a 3D printer 100 capable of independently transferring a flat plate 120 on orthogonal X-axis, Y-axis and Z-axis; a fibroblast liquid container 150 fixedly mounted on the top and storing fibroblasts differentiated from induced pluripotent stem cells (iPSCs); a keratinocyte liquid container 140 mounted adjacent to the fibroblast liquid container 150 and storing keratinocytes differentiated from the iPSCs; a first transfer pipe 145 having one end connected to the keratinocyte liquid container 140 and the other end connected to an injection device 160; a second transfer pipe 155 having one end connected to the fibroblast liquid container 150 and the other end connected to the injection device 160; and the injection device 160 having an upper part independently connected with the first and second transfer pipes 145 and 155 and having a first nozzle connected to the first transfer pipe 145 and a second nozzle connected with the second transfer pipe 155.

### TECHNICAL FIELD

The present invention relates to artificial skin using fibroblasts and keratinocytes, and more particularly, to a manufacturing device of artificial skin using cells differentiated from induced pluripotent stem cells (iPSCs).

### BACKGROUND ART

Until now, a possibility of making artificial skin by culturing fibroblasts or culturing keratinocytes from stem cells has been known through several academic papers. For this purpose, primary cultured cells are also able to be actually purchased from a commercial company (ATCC, USA).

However, these primary cultured cells have limitations in proliferation and vary greatly by object (LOT). For example, primary cultured cells once purchased could make six artificial skins through proliferation, but the primary cultured cells need to be purchased again for additional production. However, the characteristics of the artificial skins produced were not constant due to a large change in the characteristics of each primary cultured cell. This is because the change in the characteristics depends on which donor the primary cultured cells were received from.

Accordingly, the research and development of technology capable of mass-producing artificial skins having predetermined characteristics are required.

### DISCLOSURE

### Technical Problem

Therefore, the present invention has been derived to solve the problems, and an object of the present invention is to provide a manufacturing device of artificial skin using fibroblasts and keratinocytes differentiated from induced pluripotent stem cells.

Meanwhile, the technical objects to be achieved in the present invention are not limited to the aforementioned technical objects, and other technical objects, which are not mentioned above, will be apparently understood to a person having ordinary skill in the art from the following description.

### Technical Solution

According to an aspect of the present invention, there is provided a manufacturing device of artificial skin including: a 3D printer 100 capable of independently transferring a flat plate 120 on orthogonal X-axis, Y-axis and Z-axis; a fibroblast liquid container 150 fixedly mounted on the top and storing fibroblasts differentiated from induced pluripotent stem cells (iPSCs); a keratinocyte liquid container 140 mounted adjacent to the fibroblast liquid container 150 and storing keratinocytes differentiated from the iPSCs; a first transfer pipe 145 having one end connected to the keratinocyte liquid container 140 and the other end connected to an injection device 160; a second transfer pipe 155 having one end connected to the fibroblast liquid container 150 and the other end connected to the injection device 160; and the injection device 160 having an upper part independently connected with the first and second transfer pipes 145 and 155 and having a first nozzle connected to the first transfer pipe 145 and a second nozzle connected with the second transfer pipe 155.

In addition, the donor may be one person.

In addition, in the injecting step (S160), the fibroblasts and the keratinocytes may be injected in pattern forms.

In addition, the differentiating step (S140) of the fibroblasts may be performed by adding at least one of fetal bovine serum (FBS), insulin, epidermal growth factor (EGF), bone morphogenetic protein 4 (BMP4), and non essential amino acid (NEAA) in a Dulbecco's modified eagle medium (DMEM).

In addition, the DMEM may be a DMEM/F12 medium with a composition of 3:1 from the differentiation start day to day 6, a DMEM/F12 medium with a composition of 1:1 from day 7 to day 13, and a DMEM/F12 medium with a composition of 3:1 from day 14 to day 21.

In addition, the DMEM may be added with 5% of the FBS from the differentiation start day to day 21, 5 μg/ml of the insulin and 10 ng/ml of the EGF from the differentiation start day to day 6, and 5 μg/ml of the insulin and 10 ng/ml of the EGF from day 14 to day 21, 25 ng/ml of the BMP4 from day 4 to day 6, and 1% of the NEAA from day 7 to day 13, respectively.

In addition, the differentiating step (S120) of the keratinocytes may be performed by adding at least one of FBS, insulin, EGF, BMP4, NEAA, retinol acid, and CaCl₂) to the DMEM medium or dkSFM culture medium.

In addition, the DMEM may be a DMEM/F12 medium with a composition of 3:1 from the differentiation start day to day 7, and the dkSFM culture medium may be used from day 8 to day 21.

In addition, the medium may be added with 2% of the FBS from the differentiation start day to day 7, 5 μg/ml of the insulin from the differentiation start day to day 21, 25 ng/ml of the EGF from the differentiation start day to day 7, 20 ng/ml of the EGF from day 8 to day 21, 25 ng/ml of the BMP4 from the differentiation start day to day 7, 20 ng/ml of the BMP4 from day 8 to day 15, 10 ng/ml of the BMP4 from day 16 to day 21, 1 μg/ml of the retinol acid from the differentiation start day to day 15, and 1.2 mM of CaCl₂) from day 18 to day 21, respectively.

### Advantageous Effects

According to one embodiment of the present invention, since skin cells are prepared using induced pluripotent stem cells (iPSCs) donated from a single donor, there is an advantage that there is no limit to proliferation, there is a small difference in characteristics between the prepared cells, and the quality is constant.

Effects which can be obtained in the present invention are not limited to the aforementioned effects and other unmentioned effects will be clearly understood by those skilled in the art from the following description.

### DESCRIPTION OF DRAWINGS

The accompanying drawings of this specification exemplify a preferred embodiment of the present invention, the spirit of the present invention will be more clearly understood from the following detailed description taken in conjunction with the accompanying drawings, and thus it will be understood that the present invention is not limited to only contents illustrated in the accompanying drawings:

FIG. 1 is a photograph of AP staining (left) and pluripotency marker immunofluorescence staining (right) of BJ-iPSCs;

FIG. 2 is a flowchart illustrating a differentiation process of fibroblasts used in the present invention;

FIG. 3A is a photograph of human dermal fibroblasts (hDF) commercially available in ATCC;

FIG. 3B is a photograph of fibroblasts differentiated from iPSCs according to the present invention;

FIG. 4 is a graph showing a protein expression level through a vimentin marker in fibroblasts differentiated according to the present invention;

FIGS. 5A to 5C are photographs of fibroblast-specific markers and nuclei stained with various optical dyes, and FIG. 5D is a combined photograph of FIGS. 5A to 5C;

FIGS. 6A to 6E are graphs showing RNA expression levels for fibroblasts and pluripotent stem cell-specific markers;

FIG. 7 is a flowchart illustrating a differentiation process of keratinocytes used in the present invention;

FIG. 8A is a photograph of human keratinocytes commercially available in ATCC;

FIG. 8B is a photograph of keratinocytes differentiated from iPSCs according to the present invention;

FIG. 9 is a graph showing a protein expression level through a KRT14 marker in keratinocytes differentiated according to the present invention;

FIGS. 10A and 10B are photographs of keratinocyte-specific markers and nuclei stained with various optical dyes, and FIG. 10C is a combined photograph of FIGS. 10A and 10B;

FIGS. 11A to 11D are graphs showing RNA expression levels for keratinocytes and pluripotent stem cell-specific markers;

FIG. 12 is a schematic perspective view of a 3D printer according to the present invention; and

FIG. 13 is a flowchart schematically illustrating a manufacturing method of artificial skin using cells differentiated from iPSCs according to the present invention.

### BEST MODE

Hereinafter, embodiments of the present invention will be described in detail so as to be easily implemented by those skilled in the art, with reference to the accompanying drawings. A description of the present invention is merely an embodiment for a structural or functional description and the scope of the present invention should not be construed as being limited by embodiments described in a text. That is, since the embodiment can be variously changed and have various forms, the scope of the present invention should be understood to include equivalents capable of realizing the technical spirit. Further, since a specific embodiment does not necessarily mean that it should include all objects or effects or include only the effect, it should not be understood that the scope of the present invention is limited by the object or effect.

Meanings of terms described in the present invention should be understood as follows.

The terms “first”, “second”, and the like are used to differentiate a certain component from other components, but the scope of rights should not be construed to be limited by the terms. For example, a first component may be referred to as a second component, and similarly, the second component may be referred to as the first component. It should be understood that, when it is described that a component is “connected to” the other component, the component may be directly connected to the other component or another component may be present therebetween. In contrast, it should be understood that when it is described that a component is “directly connected to” the other component, another component is not present therebetween. Meanwhile, other expressions describing the relationship between the components, that is, expressions such as “between” and “directly between” or “adjacent to” and “directly adjacent to” should be similarly interpreted.

It should be understood that the singular expression encompass a plurality of expressions unless the context clearly dictates otherwise and it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

If it is not contrarily defined, all terms used herein have the same meanings as those generally understood by those skilled in the art. Terms which are defined in a generally used dictionary should be interpreted to have the same meaning as the meaning in the context of the related art, and are not interpreted as an ideal meaning or excessively formal meanings unless clearly defined in the present invention.

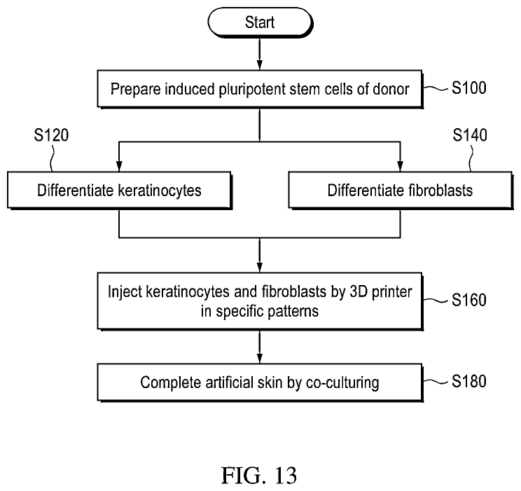

FIG. 13 is a flowchart schematically illustrating a manufacturing method of artificial skin using cells differentiated from iPSCs according to the present invention. As illustrated in FIG. 13, first, induced pluripotent stem cells (iPSCs) from a single donor are prepared (S100).

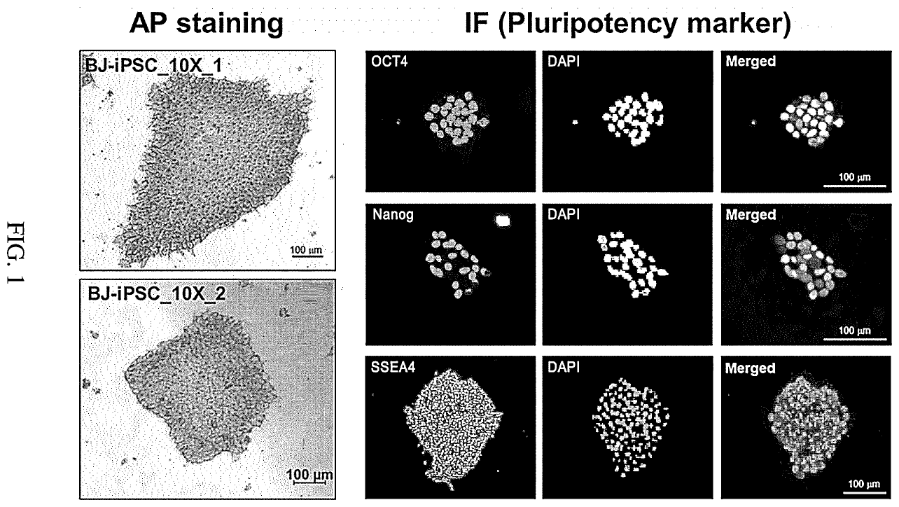

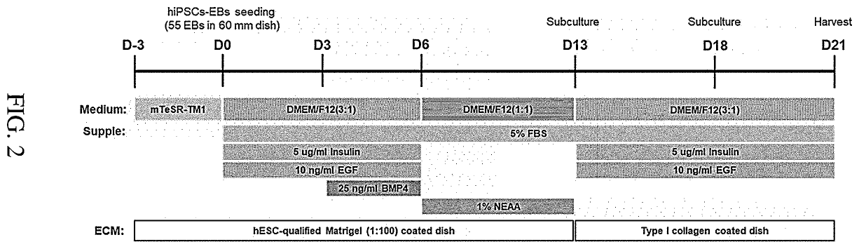

FIG. 1 is a photograph of AP staining (left) and pluripotency marker immunofluorescence staining (right) of BJ-iPSCs. As illustrated in FIG. 1, BJ fibroblast-derived human induced pluripotent stem cells (BJ-iPSC) are prepared using Episomal vectors (pCXLE-hOCT3/4-shp53-F, pCXLE-hSK, pCXLE-hUL; Addgene products) and 4D-Nucleofector technology.

In order to verify the pluripotency of reprogrammed BJ-iPSC cells, alkaline phosphatase staining is performed, and the staining is confirmed in all iPSC colonies as shown in FIG. 1. In addition, immunofluorescence staining is performed using pluripotency markers OCT4, Nanog, and SSEA4 antibodies, and it is confirmed that the markers are expressed.

Next, differentiating fibroblasts from iPSCs (S140) and differentiating keratinocytes from iPSCs (S120) are performed independently at the same time or sequentially.

### Differentiation of Fibroblasts

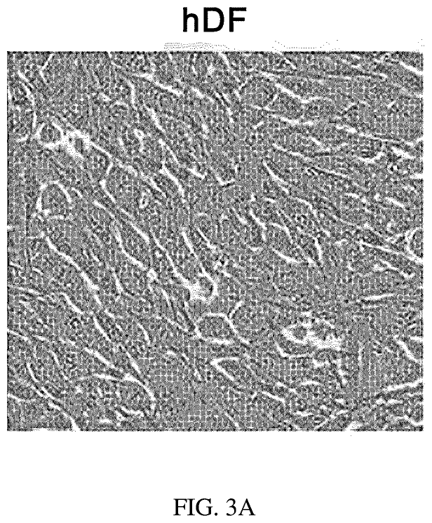

Hereinafter, the differentiating of fibroblasts (S140) will be described in detail with reference to the accompanying drawings. FIG. 2 is a flowchart illustrating a differentiation process of fibroblasts used in the present invention. As illustrated in FIG. 2, first, an mTeSR™-1 medium is prepared between day 3 (D-3) before the start of differentiation and day 1 (D-1) before the start of differentiation. The mTeSR™-1 medium is a highly specialized serum-free complete cell culture medium.

Next, on the differentiation start day (DO), a Dulbecco's modified eagle medium (DMEM) is a DMEM/F12 medium with a composition of 3:1 from the differentiation start day to day 6, a DMEM/F12 medium with a composition of 1:1 from day 7 to day 13, and a DMEM/F12 medium with a composition of 3:1 from day 14 to day 21. Specifically, 55 embryoid bodies (hiPSCs-EBs) are seeded in a 60-mm dish.

In addition, the medium is added with 5% fetal bovine serum (FBS) from the differentiation start day (D0) to day 21 (D21), 5 μg/ml of insulin and 10 ng/ml of epidermal growth factor (EGF) from the differentiation start day (D0) to day 6 (D6), 5 μg/ml of insulin and 10 ng/ml of EGF from day 14 (D14) (subculture) to day 21 (D21), 25 ng/ml of bone morphogenetic protein 4 (BMP4) from day 4 (D4) to day 6 (D6), and 1% non essential amino acid (NEAA) from day 7 (D7) to day 13 (D13), respectively. In addition, the medium is subcultured on day 18 (D18).

At this time, as an extracellular matrix (ECM), a dish coated with hESC-qualified Matrigel (1:100) is used from day 3 (D-3) before the start of differentiation (D-3) to day 12 (D12), and a dish coated with from Type I collagen is used between day 13 (D13) to day 21 (D21). Through this process, the differentiation of fibroblasts is completed on the day 21 (D21).

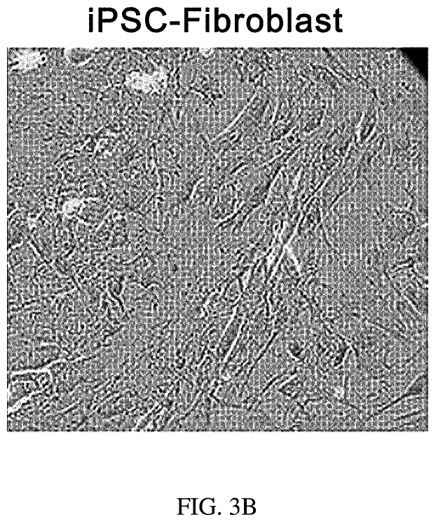

FIG. 3A is a photograph of human skin fibroblasts (hDF) commercially available in ATCC, and FIG. 3B is a photograph of fibroblasts differentiated from induced pluripotent stem cells (iPSCs) according to the present invention. When comparing FIGS. 3A and 3B, it may be confirmed that the fibroblasts (FIG. 3B) differentiated according to the present invention have a similar shape (a mesenchymal shape as a typical shape), not a complete shape to FIG. 3A while the cytoplasm is elongated.

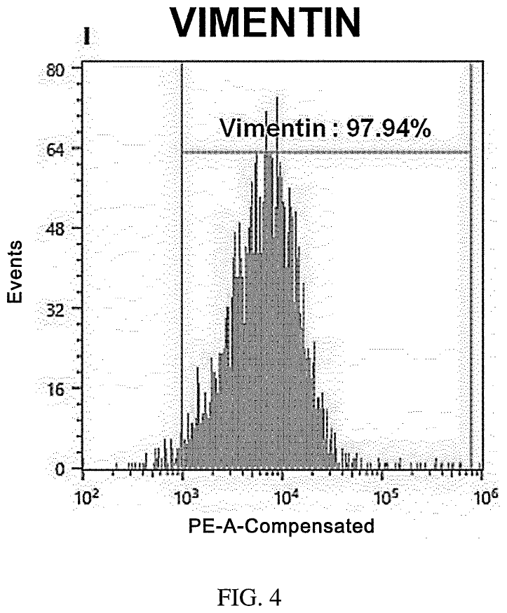

In addition, FIG. 4 is a graph showing a protein expression level through a vimentin marker in fibroblasts differentiated according to the present invention. As illustrated in FIG. 4, it can be seen that the vimentin marker is well expressed in 97.94 cells when 100 cells are differentiated. Although about 2% of the non-fibroblast state remains, it is shown that the cells have been well differentiated into highly pure fibroblasts as a whole.

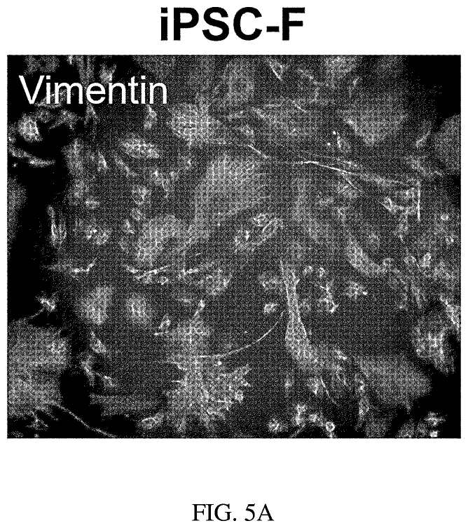

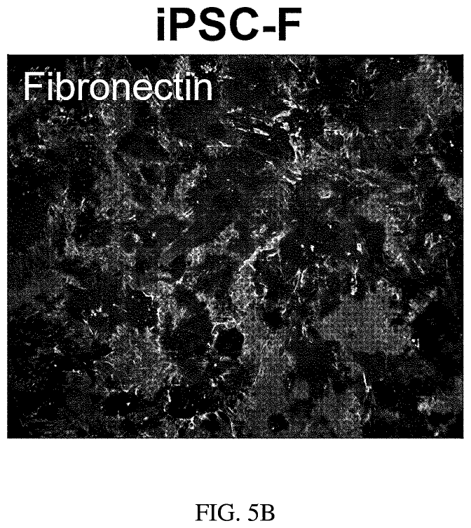

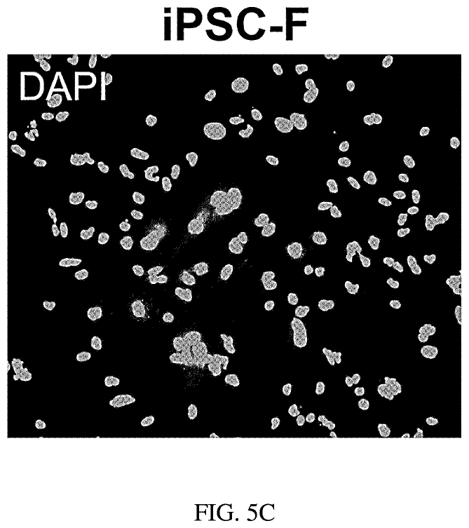

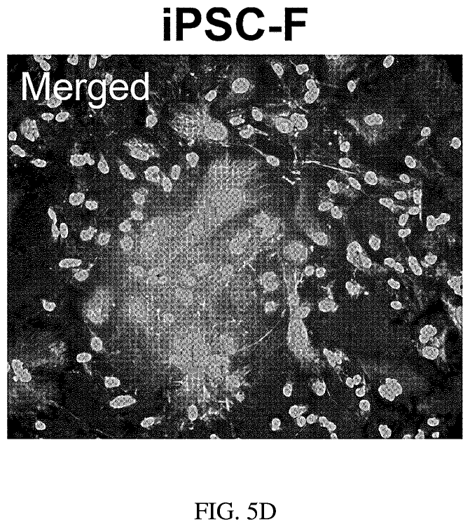

To confirm whether fibroblast markers are expressed in differentiated fibroblasts and whether specific mRNAs are expressed in differentiated fibroblasts, fluorescence staining is performed. FIGS. 5A to 5C are photographs of specific markers and nuclei of differentiated fibroblasts (iPSC-F) stained with various optical dyes, and FIG. 5D is a combined photograph of FIGS. 5A to 5C. FIG. 5A is a photograph of a vimentin marker stained with a fluorescein isothiocyanate (FITC) optical dye; FIG. 5B is a photograph of a fibronectin marker stained with a tramethylrhodamine (TRITC) optical dye; and FIG. 5C is a photograph of nuclei stained with a 4′,6-diamidino-2-phenylindole (DAPI) optical dye. It can be seen from the combined photograph of FIG. 5D that the differentiated cells are fibroblasts.

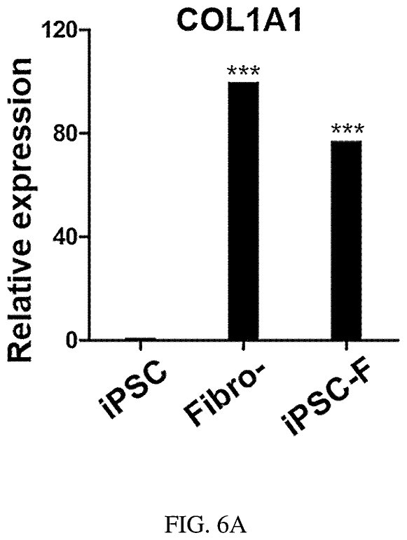

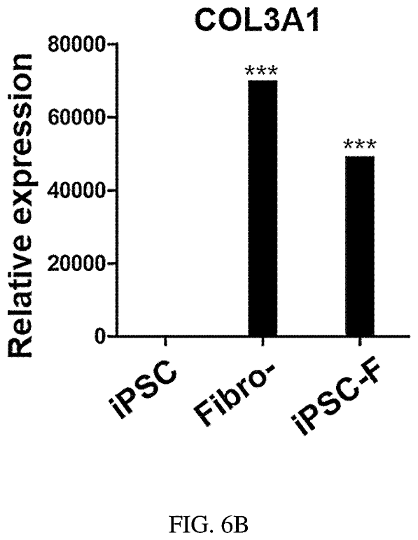

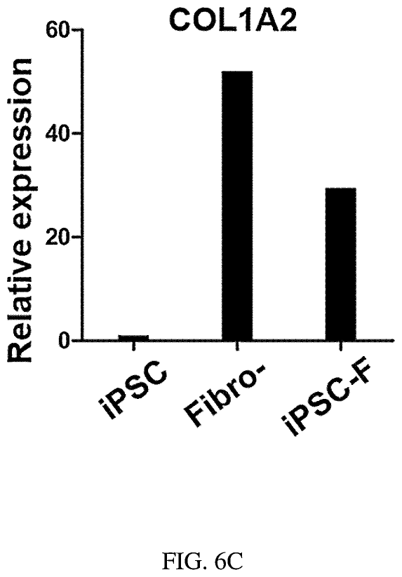

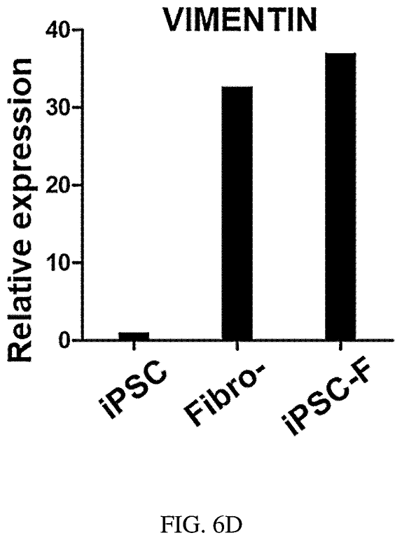

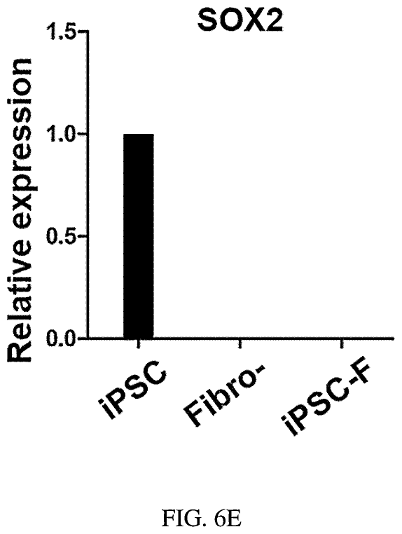

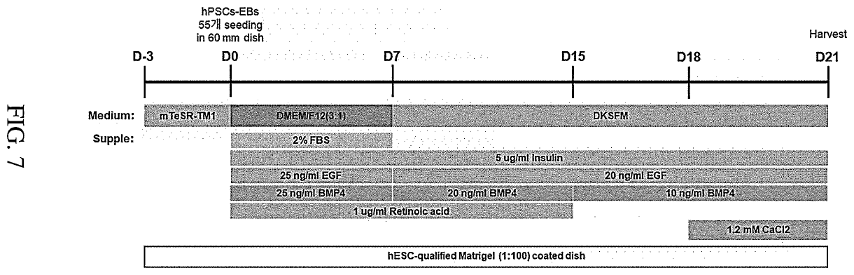

FIGS. 6A to 6E are graphs showing RNA expression levels for fibroblast-specific markers. FIG. 6A is a graph showing an expression level for a COL1A1 marker. In the case of undifferentiated iPSCs, there is no expression level thereof, and in the case of fibroblasts (Fibro-), which are primary cultured cells of an actual donor, and cells (iPSC-F) induced to differentiate into fibroblasts from induced pluripotent stem cells (iPSCs) according to the present invention, the markers are amplified 100-fold and 80-fold, respectively, resulting in high gene expression level.

FIG. 6B is a graph showing the expression level for a COL3A1 marker. In FIG. 6B, in the case of undifferentiated iPSCs, there is no expression level thereof, and in the case of fibroblasts (Fibro-), which are primary cultured cells of an actual donor, and cells (iPSC-F) induced to differentiate into fibroblasts from induced pluripotent stem cells (iPSCs) according to the present invention, the markers are amplified 70,000-fold and 50,000-fold, respectively, resulting in high gene expression level.

FIG. 6C is a graph showing the expression level for a COL1A2 marker. In FIG. 6C, in the case of undifferentiated iPSCs, there is no expression level thereof, and in the case of fibroblasts (Fibro-), which are primary cultured cells of an actual donor, and cells (iPSC-F) induced to differentiate into fibroblasts from induced pluripotent stem cells (iPSCs) according to the present invention, the markers are amplified 50-fold and 30-fold, respectively, resulting in high gene expression level.

FIG. 6D is a graph showing the expression level for a vimentin marker. In FIG. 6D, in the case of undifferentiated iPSCs, there is little expression (about 1), and in the case of fibroblasts (Fibro-), which are primary cultured cells of an actual donor, and cells (iPSC-F) induced to differentiate into fibroblasts from induced pluripotent stem cells (iPSCs) according to the present invention, the markers are amplified 33-fold and 37-fold, respectively, resulting in high gene expression level.

FIG. 6E is a graph of an SOX2 marker produced only in undifferentiated iPSCs. As illustrated in FIG. 6E, in the case of undifferentiated iPSCs, the expression level of 1.0 is exhibited, and in the case of fibroblasts (Fibro-), which are primary cultured cells of an actual donor, and cells (iPSC-F) induced to differentiate into fibroblasts from induced pluripotent stem cells (iPSCs) according to the present invention, there is no expression level. Through FIG. 6A to 6E, it can be confirmed that the differentiation of iPSCs into fibroblasts has been clearly made.

### Differentiation of Keratinocytes

Hereinafter, the differentiating of the keratinocytes (S120) will be described in detail with reference to the accompanying drawings. FIG. 7 is a flowchart illustrating a differentiation process of keratinocytes used in the present invention. As illustrated in FIG. 7, first, an mTeSR™-1 medium is prepared between day 3 (D-3) before the start of differentiation and day 1 (D-1) before the start of differentiation.

The DMEM medium is a DMEM/F12 medium with a composition of 3:1 from the differentiation start day (D0) to day 7 (D7), and a dkSFM culture medium is used from day 8 (D8) to day 21 (D21).

In addition, the medium is added with 2% FBS from the differentiation start day (D0) to day 7 (D7), 5 μg/ml of insulin from the differentiation start day (D0) to day 21 (D21), 25 ng/ml of EGF from the differentiation start day (D0) to day 7 (D7), 20 ng/ml of EGF from day 8 (D8) to day 21 (D21), 25 ng/ml of BMP4 from the differentiation start day (D0) to day 7 (D7), 20 ng/ml of BMP4 from day 8 (D8) to day 15 (D15), 10 ng/ml of BMP4 from day 16 (D16) to day 21 (D21), 1 μg/ml of retinol acid from the differentiation start day (D0) to day 15 (D15), and 1.2 mM CaCl₂) from day 18 (D18) to day 21 (D21), respectively.

At this time, as the ECM, a dish coated with hESC-qualified Matrigel (1:100) is used from day 3 (D-3) before the start of differentiation to 21 days (D21). Through this process, the differentiation of the keratinocytes is completed on day 21 (D21).

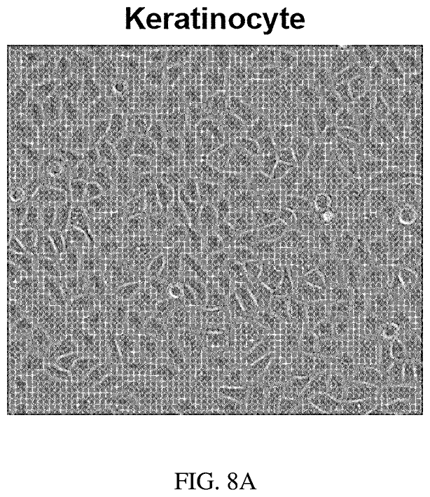

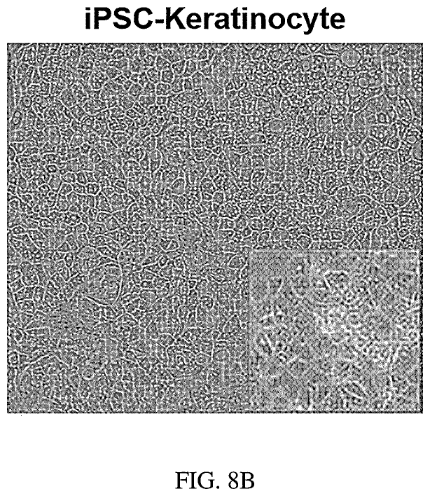

FIG. 8A is a photograph of human keratinocytes commercially available in ATCC, and FIG. 8B is a photograph of keratinocytes differentiated from iPSCs according to the present invention. As illustrated in FIGS. 8A and 8B, it can be seen that the keratinocytes (FIG. 8B) differentiated according to the present invention have a shape similar to that of FIG. 8A while the cytoplasm differentiates into a round polygonal shape.

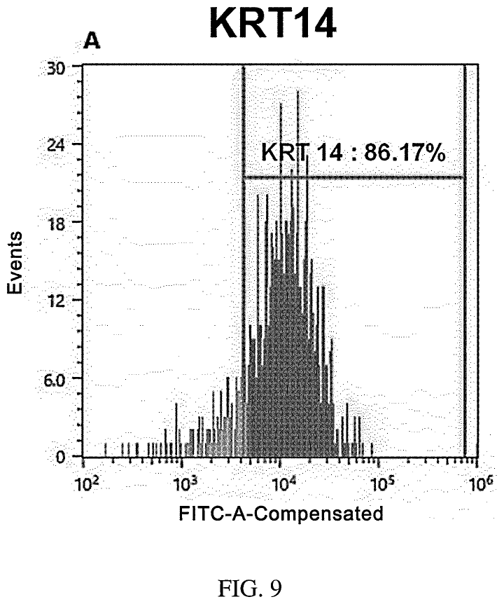

FIG. 9 is a graph showing a protein expression level through a KRT14 marker in keratinocytes differentiated according to the present invention. As illustrated in FIG. 9, it can be seen that the KRT14 marker is well expressed in 86.17 cells when 100 cells are differentiated. Although some non-keratinocytes remain, it is indicated that the cells have been well differentiated into keratinocytes as a whole.

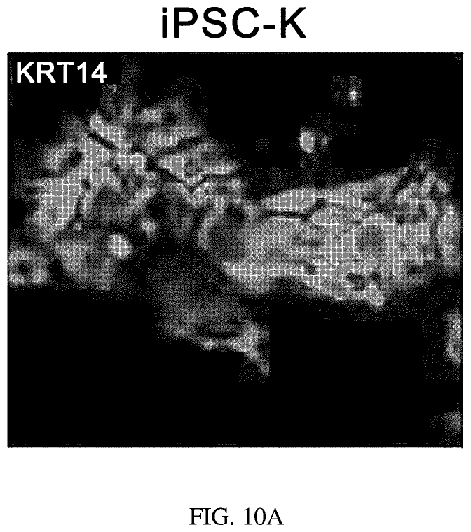

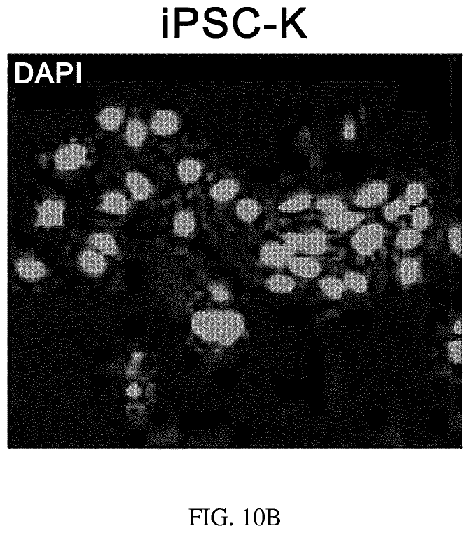

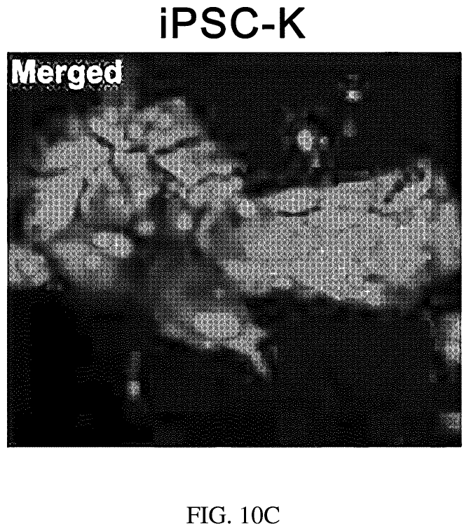

FIGS. 10A and 10B are photographs of specific markers and nuclei of respective cells stained with various optical dyes, and FIG. 10C is a combined photograph of FIGS. 10A and 10B. FIG. 10A is a photograph of the KRT14 marker stained with a TRITC optical dye, and FIG. 10B is a photograph of nuclei stained with a DAPI optical dye. It can be seen from the combined photograph of FIG. 10C that the differentiated cells are keratinocytes.

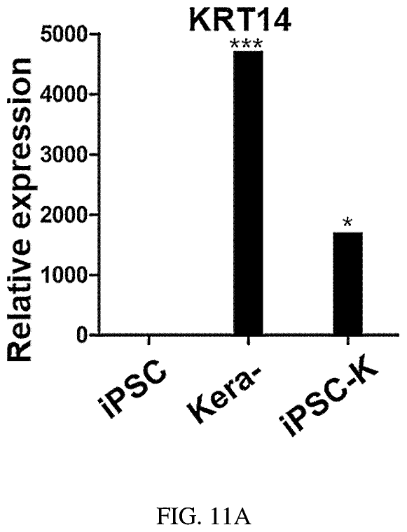

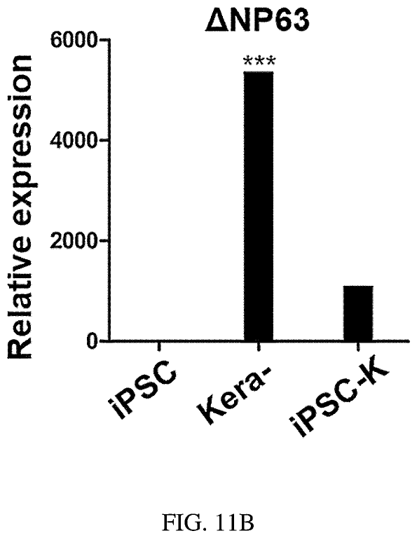

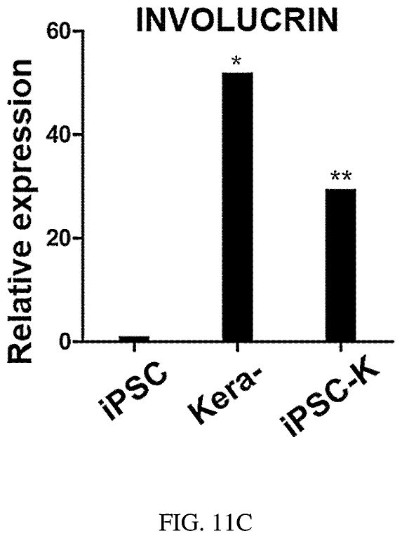

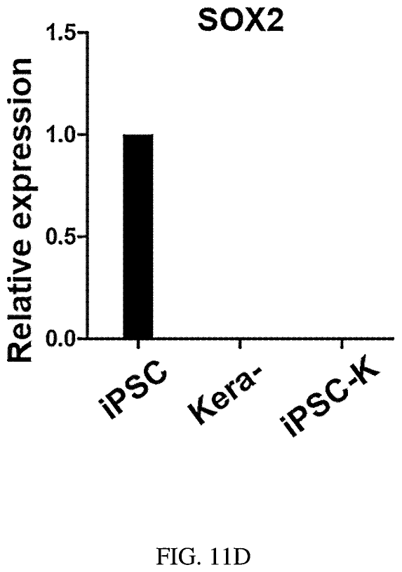

FIGS. 11A to 11D are graphs showing expression levels for keratinocyte-specific markers.

FIG. 11A is a graph showing an expression level for a KRT14 marker. In the case of undifferentiated iPSCs, there is no expression level thereof, and in the case of keratinocytes (Kera-), which are primary cultured cells of an actual donor, and cells (iPSC-K) induced to differentiate into keratinocytes from induced pluripotent stem cells (iPSCs) according to the present invention, the markers are amplified approximately 4,500-fold and 1,500-fold, respectively, resulting in high gene expression level.

FIG. 11B is a graph showing an expression level for an NP63 marker. In the case of undifferentiated iPSCs, there is no expression level thereof, and in the case of keratinocytes (Kera-), which are primary cultured cells of an actual donor, and cells (iPSC-K) induced to differentiate into keratinocytes from induced pluripotent stem cells (iPSCs) according to the present invention, the markers are amplified approximately 5,500-fold and 1,000-fold, respectively, resulting in high gene expression level.

FIG. 11C is a graph showing an expression level for an involucrin marker. In the case of undifferentiated iPSCs, very few of the expression level is shown (about 1), and in the case of keratinocytes (Kera-), which are primary cultured cells of an actual donor, and cells (iPSC-K) induced to differentiate into keratinocytes from induced pluripotent stem cells (iPSCs) according to the present invention, the markers are amplified approximately 50-fold and 30-fold, respectively, resulting in high gene expression level.

FIG. 11D is a graph of an SOX2 marker produced only in undifferentiated iPSCs. As illustrated in FIG. 11D, in the case of undifferentiated iPSCs, the expression level of 1.0 is exhibited, and in keratinocytes (Kera-), which are primary cultured cells of an actual donor, and cells (iPSC-K) induced to differentiate into keratinocytes from induced pluripotent stem cells (iPSCs) according to the present invention, there is no expression level of the marker. Through FIG. 11A to 11D, it can be confirmed that the differentiation of iPSCs into keratinocytes has been clearly made.

### MODES FOR THE INVENTION

### Preparation of Artificial Skin

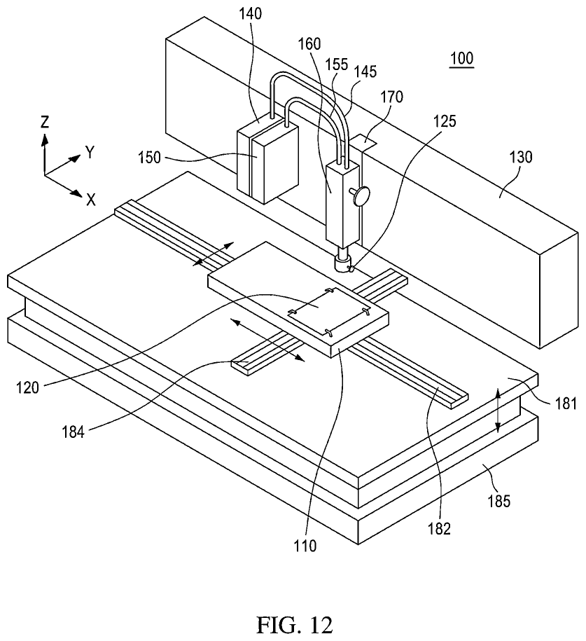

Hereinafter, a manufacturing process of artificial skin will be described in detail with reference to the accompanying drawings. Differentiated keratinocytes and fibroblasts are injected in specific patterns by a 3D printer (S160). FIG. 12 is a schematic perspective view of a 3D printer according to the present invention. As shown in FIG. 12, a transfer stage 181 is able to be reciprocally transferred in a Z-axis direction (e.g., vertical direction) by a Z-axis driver 185.

An X-axis driver 182 is installed on the transfer stage 181 to reciprocate a Y-axis driver 184 and a mounting table 110 in an X-axis direction. The Y-axis driver 184 is installed on the X-axis driver 182 to reciprocate the mounting table 110 in the Y-axis direction. To this end, servomotors and driving circuits are embedded in the X, Y, and Z-axis drivers 182, 184, and 185.

A flat plate 120 is fixed on the mounting table 110, and deposited with a keratinocyte liquid and a fibroblast liquid.

In a keratinocyte liquid container 140, the keratinocytes differentiated according to the present invention are mixed with a liquid, and in a fibroblast container 150, the fibroblasts differentiated according to the present invention are mixed with a liquid.

A first transfer pipe 145 is connected between the keratinocyte liquid container 140 and an injection device 160, and transfers the keratinocyte liquid to the injection device 160 by a suction force of a pump (not illustrated). A second transfer pipe 155 is connected between the fibroblast liquid container 150 and the injection device 160, and transfers the fibroblast liquid to the injection device 160 by the suction force of the pump (not illustrated).

First and second nozzles 125 are independently positioned at the lower end of the injection device 160 and intermittently inject the liquid toward the flat plate 120.

To implement artificial skin, the keratinocyte liquid and the fibroblast liquid may be selectively injected to form a plurality of layers. In addition, the 3D printer 100 may inject the keratinocyte liquid and the fibroblast liquid in pre-programmed specific patterns (wrinkle shapes, wavy patterns, fingerprints, etc.).

The injected flat plate 120 is co-cultured to complete artificial skin (S180).

The X, Y, and Z-axis drivers 182, 184, and 185 of the present invention may also be implemented by X, Y, and Z-axis Cartesian robots. In addition, the present invention may be used for the formation and culture of mimics of various human tissues such as corneas and tumors, artificial organs, artificial tissues, etc., in addition to artificial skin.

Detailed descriptions of the preferred embodiments of the present invention disclosed as described above are provided so as for those skilled in the art to implement and execute the present invention. The present invention has been described with reference to the preferred embodiments, but those skilled in the art will understand that the present invention can be variously modified and changed without departing from the scope of the present invention. For example, those skilled in the art may use the respective components disclosed in the embodiments by combining the respective components with each other. Therefore, the present invention is not limited to the embodiments described herein, but intends to grant the widest range which is coherent with the principles and new features disclosed herein.

The present invention may be embodied in other specific forms without departing from the spirit and essential characteristics of the present invention. Accordingly, the aforementioned detailed description should not be construed as restrictive in all terms and should be exemplarily considered. The scope of the present invention should be determined by rational construing of the appended claims and all modifications within an equivalent scope of the present invention are included in the scope of the present invention. The present invention is not limited to the embodiments described herein, but intends to grant the widest range which is coherent with the principles and new features presented herein. Further, the claims that are not expressly cited in the claims are combined to form an embodiment or be included in a new claim by an amendment after the application.

### INDUSTRIAL APPLICABILITY

The present invention relates to artificial skin using fibroblasts and keratinocytes, and more particularly, to a manufacturing device of artificial skin using cells differentiated from induced pluripotent stem cells (iPSCs).

1. A manufacturing device of artificial skin using cells differentiated from iPSCs comprising: a 3D printer (100) capable of independently transferring a flat plate (120) on orthogonal X-axis, Y-axis and Z-axis;
a fibroblast liquid container (150) fixedly mounted on a top and storing fibroblasts differentiated from the induced pluripotent stem cells (iPSCs);
a keratinocyte liquid container (140) mounted adjacent to the fibroblast liquid container (150) and storing keratinocytes differentiated from the iPSCs;
a first transfer pipe (145) having one end connected to the keratinocyte liquid container 140 and the other end connected to an injection device (160);
a second transfer pipe (155) having one end connected to the fibroblast liquid container (150) and the other end connected to the injection device (160); and
the injection device (160) having an upper part independently connected with the first and second transfer pipes (145 and 155) and having a first nozzle connected to the first transfer pipe (145) and a second nozzle connected with the second transfer pipe (155) at a lower part.

2. The manufacturing device of artificial skin using cells differentiated from iPSCs of claim 1, wherein the iPSCs are derived from a single donor.

3. The manufacturing device of artificial skin using cells differentiated from iPSCs of claim 1, wherein the fibroblasts are differentiated by adding at least one of fetal bovine serum (FBS), insulin, epidermal growth factor (EGF), bone morphogenetic protein 4 (BMP4), and non essential amino acid (NEAA) in a Dulbecco's modified eagle medium (DMEM).

4. The manufacturing device of artificial skin using cells differentiated from iPSCs of claim 3, wherein the DMEM is a DMEM/F12 medium with a composition of 3:1 from a differentiation start day to day 6,
a DMEM/F12 medium with a composition of 1:1 from day 7 to day 13, and
a DMEM/F12 medium with a composition of 3:1 from day 14 to day 21.

5. The manufacturing device of artificial skin using cells differentiated from iPSCs of claim 3, wherein the DMEM is added with 5% of the FBS from a differentiation start day to day 21,
5 μg/ml of the insulin and 10 ng/ml of the EGF from the differentiation start day to day 6, and 5 μg/ml of the insulin and 10 ng/ml of the EGF from day 14 to day 21,
25 ng/ml of the BMP4 from day 4 to day 6, and
1% of the NEAA from day 7 to day 13, respectively.

6. The manufacturing device of artificial skin using cells differentiated from iPSCs of claim 1, wherein the keratinocytes are differentiated by adding at least one of FBS, insulin, EGF, BMP4, NEAA, retinol acid, and CaCl₂) to a DMEM medium or dkSFM culture medium.

7. The manufacturing device of artificial skin using cells differentiated from iPSCs of claim 6, wherein the DMEM is a DMEM/F12 medium with a composition of 3:1 from a differentiation start day to day 7, and
the dkSFM culture medium is used from day 8 to day 21.

8. The manufacturing device of artificial skin using cells differentiated from iPSCs of claim 6, wherein the medium is added with 2% of the FBS from a differentiation start day to day 7,
5 μg/ml of the insulin from the differentiation start day to day 21,
25 ng/ml of the EGF from the differentiation start day to day 7, 20 ng/ml of the EGF from day 8 to day 21,
25 ng/ml of the BMP4 from the differentiation start day to day 7, 20 ng/ml of the BMP4 from day 8 to day 15, 10 ng/ml of the BMP4 from day 16 to day 21,
1 μg/ml of the retinol acid from the differentiation start day to day 15, and
1.2 mM of the CaCl₂) from day 18 to day 21, respectively.
